(19) United States
(12) Patent Application Publication
(10) Pub. No.: US 2014/0099256 A1
(43) Pub. Date: Apr. 10, 2014

(54) RADIOACTIVE LUMINESCENT NANOPARTICLE COMPOSITIONS

(71) Applicant: The Board of Regents of the University of Texas System, Austin, TX (US)

(72) Inventors: Jie Zheng, Allen, TX (US); Xiankai Sun, Coppell, TX (US)

(73) Assignee: The Board of Regents of the University of Texas System, Austin, TX (US)

(21) Appl. No.: 14/012,723

(22) Filed: Aug. 28, 2013

Related U.S. Application Data
(60) Provisional application No. 61/694,145, filed on Aug. 28, 2012.

(51) Int. Cl. A61K 51/12

(52) U.S. Cl. CPC A61K 51/1244
USPC 424/1.29

(57) ABSTRACT

An embodiment of the invention is directed to a composition comprising a luminescent noble metal nanoparticle, wherein the surface of the noble metal nanoparticle is coated with a ligand, and wherein the noble metal nanoparticle is about 2 nm to 5 nm in diameter and further wherein a portion of the noble metal is present as its radioactive isotope. In an embodiment of the invention, the radioactive isotope is present at a concentration of up to 2% w/w of the noble metal.

### CROSS-REFERENCES TO RELATED APPLICATIONS

This application claims the benefit under 35 U.S.C. §119(e) of U.S. Provisional Patent Application Ser. No. 61/694,145 filed Aug. 28, 2012, which is incorporated herein by reference in its entirety as if fully set forth herein.

### STATEMENT REGARDING FEDERALLY-SPONSORED RESEARCH OR DEVELOPMENT

This invention was made with government support under Grant Nos. R21EB009853 and R21EB011762 awarded by the National Institutes of Health and Grant No. RP120588 awarded by Cancer Prevention and Research Institute of Texas. The government has certain rights in the invention.

### BACKGROUND OF THE INVENTION

Early tumor detection and treatment selection is paramount to achieving therapeutic success and long-term survival rates. At its early stage, many cancers are localized and can be treated surgically. However, well-defined tumor margins are often difficult to visualize with current imaging techniques. Highly-specific, molecular-targeted probes are needed for the early detection of molecular differences between normal and tumor cells, such as cancer-specific alterations in receptor expression levels. When combined with high-resolution imaging techniques, specific molecular-targeted probes will greatly improve detection sensitivity, facilitating characterization, monitoring and treatment of cancer.

Inorganic nanoparticles (NPs) hold great potential for revolutionizing current diagnostic techniques. However, potential risks of inorganic NPs on human health remain a big challenge and require further understanding. In order to minimize the toxicity induced by the accumulation of NPs in reticuloendothelial system (RES) organs, significant efforts have been devoted to developing renal clearable nanomaterials by manipulating their sizes, shapes and surface chemistries. For instance, pioneering work on the renal clearable quantum dots (QDs) showed that the zwitterionic cysteine coated QDs with a hydrodynamic diameter (HD) of 5.5 nm could be rapidly cleared out through the urinary system within 4 h and less than ˜5% of the QDs were found in the liver. The origin of such efficient renal clearance was due to the fact that zwitterionic ligands can behave like poly(ethylene glycol) (PEG) ligands to minimize serum protein adsorption while maintaining the small HD of the QDs. Although the emergence of the new renal clearable inorganic NPs potentially further advances the translation of inorganic NPs into clinical practices, the library of renal clearable NPs is still limited. It is an objective of this invention to develop nanoprobes that possess diverse material properties suitable for different imaging techniques and also exhibit optimal in vivo pharmacokinetics.

### SUMMARY OF THE INVENTION

An embodiment of the invention is directed to a composition comprising a luminescent noble metal nanoparticle, wherein the surface of the noble metal nanoparticle is coated with a ligand, and wherein the noble metal nanoparticle is about 2 nm to 5 nm in diameter and further wherein a portion of the noble metal is present as its radioactive isotope. In an embodiment of the invention, the radioactive isotope is present at a concentration of up to 2% w/w of the noble metal.

### BRIEF DESCRIPTION OF THE DRAWINGS

FIGS. 1A to 1D show the biodistribution and pharmacokinetics of GS-[¹⁹⁸Au]AuNPs in normal Balb/c mice. (A) The time-activity-curve (TAC) of GS-[¹⁹⁸Au]AuNPs in the blood. The pharmacokinetic parameters were determined by fitting the data with a two-compartment model. (B) Excretion of GS-[¹⁹⁸Au]AuNPs in the urine at 1, 4, 2, and 48 h p.i; (C) TACs of GS-[¹⁹⁸Au]AuNPs in liver, spleen, intestines and kidneys (inset); (D) Biodistribution of GS-[¹⁹⁸Au]AuNPs. (Bld: blood, Hrt: heart, Kdy: kidney, Spl: spleen, Stm: stomach, Msl: muscle, S.I.: small intestine, L.I.: large intestine); and

FIGS. 2A to 2E show (A) Ex vivo fluorescent images of urine of control mice or mice injected with luminescent GS-CuNPs (λex=510 nm, λem=790 nm); (B) Copper contents (n=6) in the urine at different times post injection (p.i.); (C) Biodistribution of Cu(II)-GSSG complex and luminescent GS-CuNPs in BALB/c mice (n=6) 24 h after the tail-vein injection; (D) Liver-tourine ratios of Cu(II)-GSSG complex and GS-CuNPs at 24 h p.i; (E) Pharmacokinetics (n=3) of the renal clearable Cu(II)-GSSG complex and luminescent GS-CuNPs in 24 h p.i.

### DETAILED DESCRIPTION OF EXEMPLARY EMBODIMENTS

The claimed invention provides nanoparticle compositions comprising a luminescent nanoparticle, methods for preparing the nanoparticle compositions and methods of using the nanoparticle compositions. The compositions of the present invention comprise noble metal nanoparticles, which are capable of emitting in the near infrared region of the light spectrum. The claimed invention provides for compositions comprising a luminescent noble metal nanoparticle. In an embodiment, the noble metal nanoparticle comprises between 2 and 1000 noble metal atoms. In preferred embodiments, the noble metal is selected from the group consisting of gold, silver, and copper.

In certain embodiments of the compositions of the claimed invention, a portion of the noble metal is present as its radioactive isotope. In an embodiment of the invention, the radioactive isotope is present at a concentration of up to 2% w/w of the noble metal. Embodiments of the invention comprise the radioactive isotopes of noble metal. In certain embodiments the radioactive isotope is ¹⁹⁸Au. In other embodiments, the radioactive isotope is ⁶⁴Cu. The presence of the radioactive isotope in the metal nanoparticle aids in the rapid monitoring of the pharmacokinetics of the NIR emitting radioactive particles and also offers an opportunity for in vivo SPECT imaging by emitting gamma rays.

The properties of the nanoparticles enable excretion through the kidneys, as well as selective uptake and retention in tumors compared with normal tissues. This, along with the lack of in vivo toxicity, has resulted in a composition that is promising for translation to the clinic.

In certain embodiments of the claimed invention, the surface of the luminescent noble metal nanoparticle is modified with a ligand. In certain embodiments, the surface of the nanoparticle is coated with a ligand that prevents adsorption of serum proteins on the surface of the nanoparticle and prevents fouling of the nanoparticle. In certain embodiments of the invention, the surface ligand is a protein or peptide. In other embodiments of the invention, the ligand is a polymer such as poly(ethylene glycol) (PEG) or oligo(ethylene glycol) (OEG). In certain embodiments of the invention, the anti-fouling ligand is a zwitterionic material such as sulfobetaine methacrylate (SBMA), carboxybetaine methacrylate (CBMA), poly(carboxybetaine acrylamide) (polyCBAA) or a mixed charge material. In certain embodiments of the invention the ligand is glutathione.

In certain embodiments, the ligand is capable of binding to at least one cellular component. The cellular component may be associated with specific cell types or having elevated levels in specific cell types, such as cancer cells or cells specific to particular tissues and organs. Accordingly, the nanoparticle can target a specific cell type, and/or provides a targeted delivery for the treatment and diagnosis of a disease. As used herein, the term “ligand” refers to a molecule or entity that can be used to identify, detect, target, monitor, or modify a physical state or condition, such as a disease state or condition. For example, a ligand may be used to detect the presence or absence of a particular receptor, expression level of a particular receptor, or metabolic levels of a particular receptor. The ligand can be, for example, a peptide, a protein, a protein fragment, a peptide hormone, a sugar (i.e., lectins), a biopolymer, a synthetic polymer, an antigen, an antibody, an antibody fragment (e.g., Fab, nanobodies), an aptamer, a virus or viral component, a receptor, a hapten, an enzyme, a hormone, a chemical compound, a pathogen, a microorganism or a component thereof, a toxin, a surface modifier, such as a surfactant to alter the surface properties or histocompatability of the nanoparticle or of an analyte when a nanoparticle associates therewith, and combinations thereof.

The present invention further encompasses methods of using the luminescent nanoparticles in order to study a biological state. The invention provides for a method of monitoring a molecule of interest by contacting the luminescent noble metal nanoparticle with a sample containing the molecule of interest. In a preferred embodiment, the molecule of interest is present in a biological sample.

Unless otherwise noted, the terms used herein are to be understood according to conventional usage by those of ordinary skill in the relevant art. It is to be understood that as used in the specification and in the claims, “a” or “an” can mean one or more, depending upon the context in which it is used. Thus, for example, reference to “a cell” can mean that at least one cell can be utilized.

The present invention may be understood more readily by reference to the following detailed description of the preferred embodiments of the invention and the Working Examples included herein. However, before the present compounds, compositions, and methods are disclosed and described, it is to be understood that this invention is not limited to specific noble metals, specific conditions, or specific methods, etc., as such may, of course, vary, and the numerous modifications and variations therein will be apparent to those skilled in the art. It is also to be understood that the terminology used herein is for the purpose of describing specific embodiments only and is not intended to be limiting.

As used herein, the term “noble metal” refers to the group of elements selected from the group consisting of gold, silver, and copper and the platinum group metals (PGM) platinum, palladium, osmium, iridium, ruthenium and rhodium. In certain preferred embodiments of the present invention, the noble metal is selected from the group consisting of gold, silver, and copper. In other preferred embodiments, the noble metal is silver. In other preferred embodiments, the noble metal is gold. In other preferred embodiments, the noble metal is copper.

As used herein, the term “nanoparticle” refers to an association of 2-1000 atoms of a metal. Nanoparticles may have diameters in the range of about 2 to about 5 nm. In other preferred embodiments, the nanoparticles comprise approximately 2-1000, approximately 2-500, approximately 2-250, approximately 2-100, approximately 2-25 atoms, or approximately 2-10 atoms.

Compositions of the invention are capable of emitting in the near infra-red range of 700 nm to 1100 nm. Compositions of the claimed invention are capable of being detected by Single-photon emission computed tomography (SPECT) and fluorescence imaging techniques. Therefore, the nanoprobes of the claimed invention can serve as dual-modality imaging probes.

The present invention further encompasses methods for the preparation of the noble metal nanoparticle having the characteristics as described herein. In one embodiment, the method of preparing a noble metal nanoparticle comprises the steps of: a) combining an aqueous solution comprising a noble metal, and an aqueous solvent to create a combined solution; b) adding a first ligand; c) mixing the combined solution to allow the formation of a noble metal nanoparticle; and d) adjusting the pH of the combined solution using acid or base. In certain embodiments of the invention, the aqueous solution of the noble metal contains an amount of a radioactive isotope of the noble metal. In an embodiment of the invention, the radioactive isotope is present in the nanoparticle at a concentration of up to 2% w/w of the noble metal.

In certain embodiments of these methods, a reducing agent is added to the combined solution to reduce the noble metal nanoparticle. Preferably the reducing agent is selected from the group comprising a chemical reducing agent, light, or a combination thereof. In certain embodiments of these methods, light can be used as a reducing agent to photoreduce the noble metal nanoparticles. In certain other embodiments of these methods, a chemical reducing agent can be used as a reducing agent. In one embodiment, light is used in combination with a reducing agent to photoreduce the noble metal nanoparticles.

Preferably, the aqueous solution comprising a noble metal ion used in the preparation of the compounds is selected from the group consisting of AgNO₃, HAuCl₄.nH₂O, and CuCl₂.nH₂O. In one embodiment, the aqueous solution comprising a noble metal is AgNO₃.

In another embodiment, the aqueous solution comprising a noble metal is HAuCl₄.nH₂O. In a further embodiment, the aqueous solution comprising a noble metal is CuCl₂.nH₂O.

In one embodiment, the aqueous solution comprising a noble metal is HAuCl₄.nH₂O, a reducing agent is added to the combined solution along with a ligand, the pH adjusted, and the combined solution is mixed for at least one hour to allow the formation of the gold nanoparticle. In another embodiment, the pH adjusted, combined solution is mixed for about 48 hours or longer (up to several months) to allow the formation of a luminescent gold nanoparticle. In another embodiment, noble metal nanoparticles are created through photoreduction through irradiation with visible or ultraviolet light to allow the formation of a gold, silver or copper nanoparticle.

In an embodiment of the invention, a therapeutic agent is attached to the nanoparticle. The therapeutic agent is selected from the group consisting of antibiotics, antimicrobials, antiproliferatives, antineoplastics, antioxidants, endothelial cell growth factors, thrombin inhibitors, immunosuppressants, anti-platelet aggregation agents, collagen synthesis inhibitors, therapeutic antibodies, nitric oxide donors, antisense oligonucleotides, wound healing agents, therapeutic gene transfer constructs, extracellular matrix components, vasodialators, thrombolytics, antimetabolites, growth factor agonists, antimitotics, statin, steroids, steroidal and nonsteroidal anti-inflammatory agents, angiotensin converting enzyme (ACE) inhibitors, free radical scavengers, PPAR-gamma agonists, small interfering RNA (siRNA), microRNA, and anti-cancer chemotherapeutic agents.

In an embodiment of the invention, the compositions of the invention are used to monitor the surface of cell membranes. In certain embodiments of the invention, the cell membranes that are targeted are tumor cell membranes.

After administration of the nanoparticle to a subject, the blood residence half-time of the nanoparticles may range from about 2 hours to about 25 hours, from about 3 hours to about 20 hours, from about 3 hours to about 15 hours, from about 4 hours to about 10 hours, or from about 5 hours to about 6 hours. Longer blood residence half-time means longer circulation, which allows more nanoparticles to accumulate at the target site in vivo. Blood residence half-time may be evaluated as follows. The nanoparticles are first administered to a subject (e.g., a mouse, a miniswine or a human). At various time points post administration, blood samples are taken to measure nanoparticle concentrations through suitable methods.

An embodiment of the claimed invention is directed to a method for detecting a component of a cell comprising the steps of: contacting the cell with a composition comprising a luminescent noble metal nanoparticle, wherein the surface of the noble metal nanoparticle is coated with a ligand, and wherein the noble metal nanoparticle is about 2 nm to 5 nm in diameter, wherein a portion of the noble metal is present as a radionuclide; and monitoring the binding of the nanoparticle to the cell or a cellular component by at least one imaging technique.

A further embodiment of the invention is directed to a method for targeting a tumor cell comprising administering the tumor cell an effective amount of a composition comprising a luminescent noble metal nanoparticle, wherein the surface of the noble metal nanoparticle is coated with a ligand, and wherein the noble metal nanoparticle is about 2 nm to 5 nm in diameter, wherein a portion of the noble metal is present as a radionuclide, wherein the ligand is capable of binding a tumor marker; and at least one therapeutic agent.

An embodiment of the claimed invention is directed to a metal nanoparticle that is renal clearable. In certain embodiments of the invention, the compositions demonstrate greater than 50% renal clearance within 48 hours of administration.

### WORKING EXAMPLES

Glutathione-Coated Luminescent Gold Nanoparticles (G-AuNPs) were synthesized by a self-dissociation of Au(I)-glutathione polymers in an aqueous solution. In a typical synthesis, a fresh aqueous solution containing reduced L-glutathione (25 mM) was added into a HAuCl₄ aqueous solution (25 mM) containing ¹⁹⁸Au at a 1:1 molar ratio of gold to thiolated ligand. The solution was centrifuged at 21,000 g for 1 minute to remove the insoluble aggregates as well as large NPs. The supernatant was further purified by adding a small amount of ethanol into the aqueous solution (the ratio between water and ethanol is 2:1). Under such conditions, the luminescent gold NPs were precipitated out of the solution while the free GSH and gold ions remained in the solution. The precipitates were then re-suspended in aqueous solution (DI water, PBS or 10% (v/v) FBS-containing MEM (without phenol red). The final solution contained G-AuNPs with diameter of ˜2 nm. The pH of solution was adjusted by 1 M NaOH or 1 M HCl and then measured by a pH meter.

CuNPs were synthesized by mixing 1 mmol ⁶⁴CuCl₂ and 4 mmol glutathione (GSH) in 40 mL de-ionized (DI) water, followed by adding 5 M NaOH solution to tune the pH of the mixture to about 7, and adding extra DI water to fix the total volume to 50 mL, which was then equally divided into 10 samples in 20 mL vials, which were closed and stored at room temperature for about one week till the solution color unchanged, and the blue Cu(II)-GSSG complex was obtained. The synthesized ⁶⁴Cu(II)-GSSG complex solution of 4.8 mL was precipitated by adding 3 times volume ethanol (21,000 g, 1 min). Then, the precipitates were dried by N₂ purge and redissolved in 45 mL DI water, followed by adding fresh prepared 2 mL 4.8 M NaBH₄ solution under stirring with the speed of 1150 rpm for 5-10 min. Then, the pH of the solution was tuned to 4-5 by adding 5 M HCl solution, and the solution was kept at R.T. for ˜30 min under stirring to form ⁶⁴Cu radioactive luminescent nanoparticles.

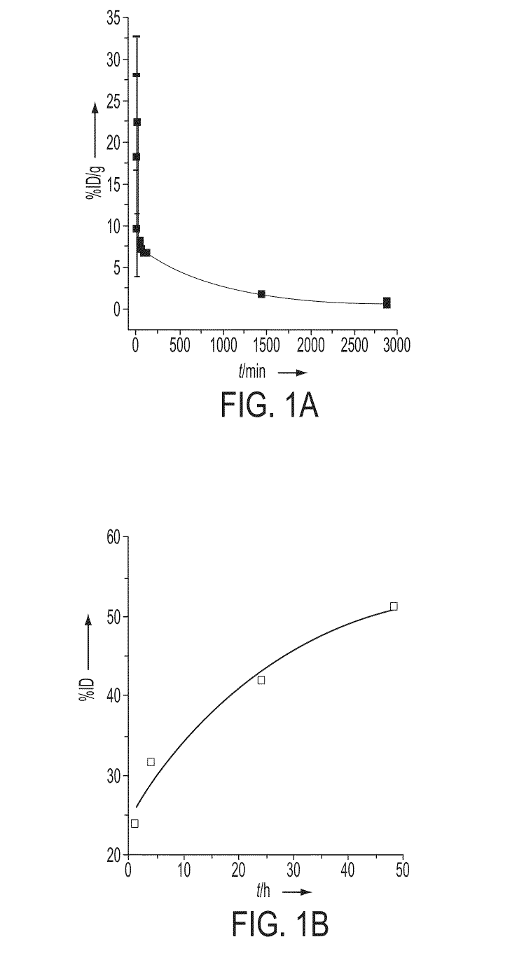

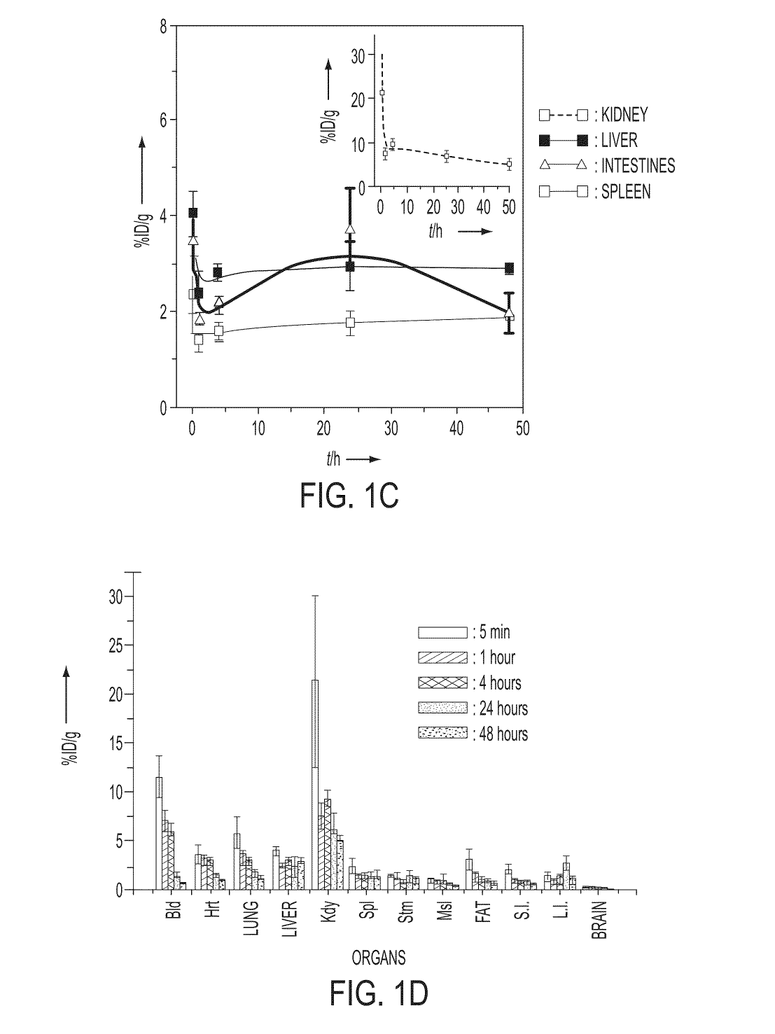

The pharmacokinetic parameters of GS-[¹⁹⁸Au]AuNPs were measured in normal Balb/c mice. The results showed that the GS-[¹⁹⁸Au]AuNPs exhibited a two compartment profile of in vivo kinetics with a t½, of 5.0 min and a t½_{β} of 12.7 hours (FIG. 1A). The short t½_{α} of GS-[¹⁹⁸Au]AuNPs indicates that these NPs can rapidly distribute into tissues during the circulation, but different from NPs-based agents such as bovine serum albumin-coated and mercaptoundecanoic acid-coated quantum dots (QDs) with a single half-life of ˜0.65 h and ˜0.98 h, respectively. The relatively long t½_{β} of GS-[¹⁹⁸Au]AuNPs is comparable to the t½β (˜17.2 h) of ⁶⁴Cu-labeled DOTA-NHGR11, further indicating that GS-[¹⁹⁸Au]AuNPs indeed behaved like small molecular probes in pharmacokinetics.

More than 50% of GS-[¹⁹⁸Au]AuNPs were cleared out of the body after 48 hours (FIG. 1B), indicating that these radioactive NIR emitting AuNPs are renal clearable. Biodistributions of GS-[¹⁹⁸Au]AuNPs in kidney, liver, spleen and intestines at 5 min., 1, 4, 24, 48 hours after IV injection also provide additional insights on the clearance pathways and kinetics (FIG. 1C). While the pharmacokinetic profile of the NPs varies with organs, one common feature is that the highest accumulation of the NPs occurred within 5 min. p.i. followed by efficient clearance. This observation is consistent with the measured short t½_{α}, indicating that the tissue distribution of the NPs was indeed rapid. The highest uptake of GS-[¹⁹⁸Au]AuNPs in the kidney was followed by a gradual decrease from ˜21% ID/g to ˜5% ID/g (inset of FIG. 1C), which was in tandem with the clearance of the NPs from the blood (FIG. 1A), indicating that the NPs were mainly excreted through the glomerular filtration. While the rapid decreases of the particle concentrations in the liver, spleen and intestines in the first hour was due to reentrance of the NPs into the blood, these organs indeed provide additional clearance pathways. Interestingly, the accumulations of the NPs in the liver and spleen roughly remained constant even though the NPs were still circulating in the blood, indicating the slow clearance of the NPs through hepatic route. The uptake level of the GS-[¹⁹⁸Au]AuNPs in intestines varied with time, reaching its maxima (˜3.7% ID/g) at ˜24 hours p.i. and then decreasing to ˜1.9% ID/g after 48 p.i., suggesting that small amount of GS-[¹⁹⁸Au]AuNPs can be cleared out of the body through the metabolism route in addition to the urinary system. The detailed biodistribution data of the GS-[¹⁹⁸Au]AuNPs in other organs are summarized in FIG. 1D.

The accumulation of the NPs in heart, lung, muscle and fat also decreased with time, indicating that the GS-[¹⁹⁸Au]AuNPs were only temporally distributed to these tissues due to the rapid adsorption process and can be efficiently washed away within the first 48 hours without causing an uptake increase in the liver and spleen. The origin of the resulted biodistribution and efficient renal clearance of the NPs were attributed to the small particle size and glutathione ligand, which enable the GS-[¹⁹⁸Au]AuNPs highly stable in the physiological environment and resistant to serum protein adsorption. As a result, the hydrodynamic diameter of the NPs in the body was still below the size threshold for renal clearance and the NPs remained stealthy to RES organs.

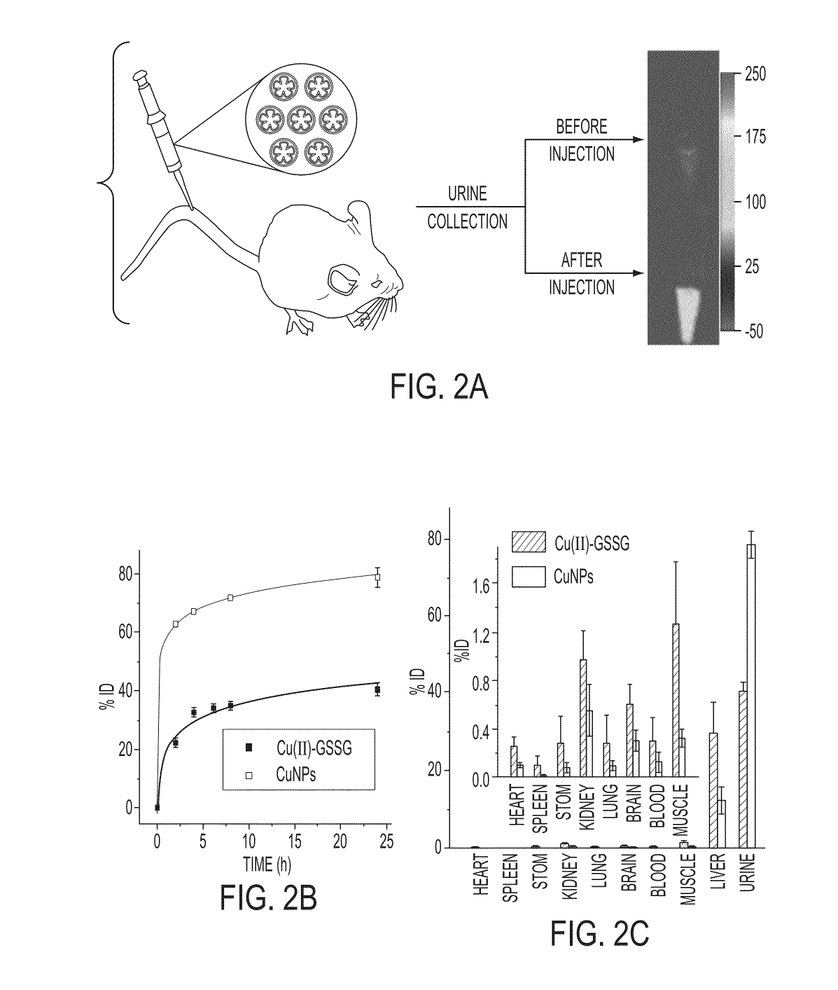

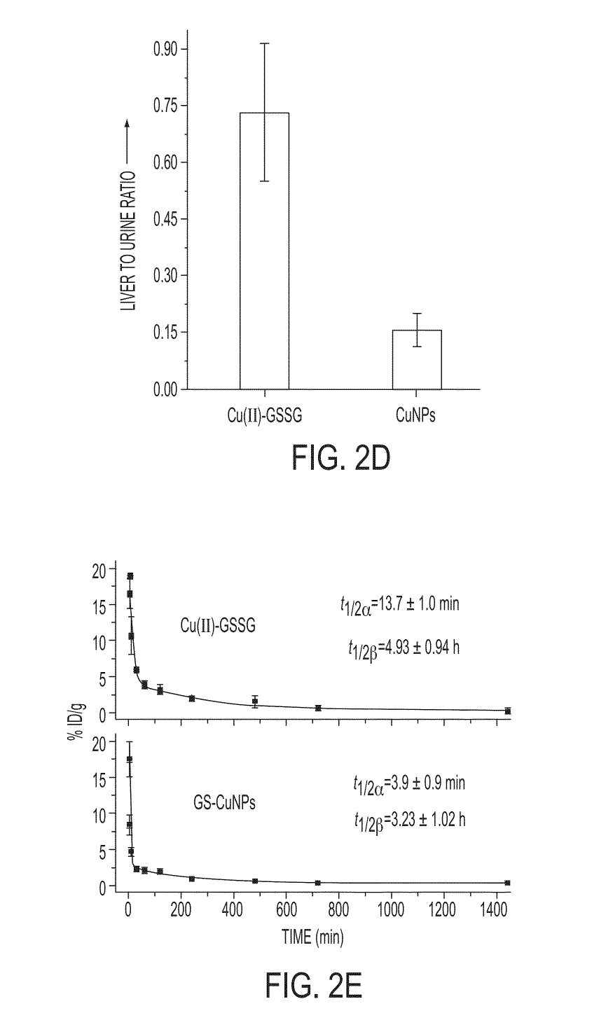

Although “being small” is generally seen as an efficient approach to boost the clearance of biomaterials from the body, the avidity between materials and serum proteins also plays an important role in the excretion. Seemingly a dilemma, it is hard to know which factors in the renal excretion of GS-CuNPs and Cu(II)-GSSG complex are reliably important. As such, the renal clearance kinetics and biodistribution of GS-CuNPs and Cu(II)-GSSG complex were evaluated in BALB/c mice (FIGS. 2A and 2B). For GS-CuNPs, 78.5±3.5% of injected copper was excreted from the body into the urine after 24 h p.i., and over 60% of copper was actually eliminated from the body in the first 2 h p.i (FIG. 2B). Under the same conditions, the Cu(II)-GSSG complexes were also renal clearable, however, their clearance efficiency was much lower than GS-CuNPs, only −22% ID of copper were found in the urine of the mice injected with Cu(II)-GSSG at 2 h p.i. Interestingly, although GS-CuNPs were much larger than Cu(II)-GSSG complexes, GS-CuNPs were even more efficiently cleared out of the body than the complexes in the first 2 h p.i. Additional increases in urine excretion of copper metal for GS-CuNPs (−16%) and Cu(II)-GSSG complexes (−18%) after 2 h are comparable (FIG. 2B), implying that the major differences in renal clearance efficiencies among the particles and complexes originated from very initial clearance stage before the dissociation of GS-CuNPs into Cu(II)-GSSG complex at a later stage with the presence of proteins.

The detailed biodistribution studies of mice injected with GS-CuNPs and Cu(II)-GSSG complexes were consistent with their renal clearance profiles (FIG. 2C). In view of the background of copper, the average copper contents in the various organs/tissues and urine from three BALB/c mice have been deducted. The rapid clearance of GS-CuNPs significantly reduced their nonspecific accumulation of the GS-CuNPs in RES organs, and only 12.3±3.1% and −0.03% ID of copper was found in the liver and spleen, respectively. In contrast, Cu(II)-GSSG complex showed remarkably higher accumulation in the organs/tissues (29.6±8.1% in the liver, ˜0.1% in the spleen) than GS-CuNPs. However, compared to free Cu ions that often bind to caeruloplasmin and hephaestin and mainly accumulate in the liver, both GS-CuNPs and Cu(II)-GSSG complexes had much lower accumulation in RES, which was because GSH minimized the serum protein absorption and render them the efficient renal clearance. In addition, the more effective renal clearance and lower accumulation in RES organs of GS-CuNPs than that of Cu(II)-GSSG complexes can be ascribed to the −20% binding of Cu(II)-GSSG complexes to the serum proteins at physiological environment. As a result, liver-to-urine ratio (−0.16) of CuNPs is more than 4 times lower than that of Cu(II)-GSSG complex (FIG. 2D). The observed low accumulation of GS-CuNPs was also supported by the very short blood elimination half-lives of GS-CuNPs (FIG. 2E), which exhibit a rapid distribution with t½α of 3.9±0.9 min and terminal elimination half-life (t½β) of 3.23±1.02 h, similar to many small molecules and ˜4 times shorter than GS-AuNPs. Meanwhile, a slightly slower distribution half-life (t½α=13.7±1.0 min) and relatively longer terminal elimination half-life t½β=4.93±0.94 h) of Cu(II)-GSSG complex supported the observation in biodistribution and renal clearance, where the copper contents in the organs or tissues were much higher than that of GS-AuNPs.

The successful synthesis of renal clearable GS-CuNPs provided an exciting opportunity to explore a potential biomedical application of CuNPs in PET imaging because ⁶⁴Cu is a well known β⁺ (0.653 MeV, 17.8%) emitter for PET imaging with a half-time of 12.7 h. In addition, considering copper is a necessary trace element in human body with the total content of 100-150 mg and ˜18 mg in the liver the trace amount of renal clearable CuNPs can be combined with highly sensitive nuclear imaging techniques, potential side effect induced by copper metal in the liver can be further minimized.

Although the present invention has been described in connection with some embodiments, it is not intended to be limited to the specific form set forth herein. Rather, the scope of the present invention is limited only by the accompanying claims. Additionally, although a feature may appear to be described in connection with particular embodiments, one skilled in the art would recognize that various features of the described embodiments may be combined in accordance with the invention. In the claims, the term comprising does not exclude the presence of other elements or steps.

Furthermore, although individually listed, a plurality of means, elements or method steps may be implemented by e.g. a single unit or processor. Additionally, although individual features may be included in different claims, these may possibly be advantageously combined, and the inclusion in different claims does not imply that a combination of features is not feasible and/or advantageous. Also, the inclusion of a feature in one category of claims does not imply a limitation to this category but rather indicates that the feature is equally applicable to other claim categories as appropriate. Furthermore, the order of features in the claims do not imply any specific order in which the features must be worked and in particular the order of individual steps in a method claim does not imply that the steps must be performed in this order. Rather, the steps may be performed in any suitable order. In addition, singular references do not exclude a plurality. Thus references to “a”, “an”, “first”, “second” etc do not preclude a plurality.

What is claimed is:

1. A composition comprising a luminescent noble metal nanoparticle, wherein the surface of the noble metal nanoparticle is coated with a ligand, and wherein the noble metal nanoparticle is about 2 nm to 5 nm in diameter, wherein a portion of the noble metal is present as a radionuclide.

2. The composition of claim 1, wherein the radionuclide is present at a concentration of up to 2% w/w of the noble metal.

3. The composition of claim 1, wherein the noble metal is selected from the group consisting of gold, silver, and copper.

4. The composition of claim 1, wherein the ligand is a protein, polymer, zwitterionic material or a mixed charge material.

5. The composition of claim 4, wherein the ligand is glutathione.

6. The composition of claim 1, wherein the ligand is capable of binding to at least one cellular component.

7. The composition of claim 6, wherein the cellular component is a tumor marker.

8. The composition of claim 1, wherein the nanoparticle is detectable by PET, SPECT, CT, MRI, optical imaging, bio luminescence imaging, or combinations thereof.

9. The composition of claim 8, wherein the optical imaging is fluorescence imaging.

10. The composition of claim 8, wherein the optical imaging is near infrared imaging.

11. The composition of claim 1, wherein a therapeutic agent is attached to the nanoparticle.

12. The composition of claim 11, wherein the therapeutic agent is selected from the group consisting of antibiotics, antimicrobials, antiproliferatives, antineoplastics, antioxidants, endothelial cell growth factors, thrombin inhibitors, immunosuppressants, anti-platelet aggregation agents, collagen synthesis inhibitors, therapeutic antibodies, nitric oxide donors, antisense oligonucleotides, wound healing agents, therapeutic gene transfer constructs, extracellular matrix components, vasodialators, thrombolytics, antimetabolites, growth factor agonists, antimitotics, statin, steroids, steroidal and nonsteroidal anti-inflammatory agents, angiotensin converting enzyme (ACE) inhibitors, free radical scavengers, PPAR-gamma agonists, small interfering RNA (siRNA), microRNA, and anti-cancer chemotherapeutic agents.

13. The composition of claim 1, wherein blood residence half-time of the nanoparticle after administration of the nanoparticle to a subject ranges from about 2 hours to about 25 hours.

14. The composition of claim 13, wherein the blood residence half-time of the nanoparticle ranges from about 3 hours to about 15 hours.

15. The composition of claim 13, wherein the blood residence half-time of the nanoparticle ranges from about 4 hours to about 10 hours.

16. The composition of claim 1, wherein renal clearance of the nanoparticle after administration of the nanoparticle to a subject is greater than 50% in about 48 hours.

17. A method for detecting a component of a cell comprising the steps of:
contacting the cell with a composition comprising a luminescent noble metal nanoparticle, wherein the surface of the noble metal nanoparticle is coated with a ligand, and wherein the noble metal nanoparticle is about 2 nm to 5 nm in diameter, wherein a portion of the noble metal is present as a radionuclide; and monitoring the binding of the nanoparticle to the cell or a cellular component by at least one imaging technique.

18. The method of claim 17, wherein the imaging technique is selected from the group consisting of PET, SPECT, CT, MRI, optical imaging, bio luminescence imaging, and combinations thereof.

19. A method for targeting a tumor cell comprising administering to the tumor cell an effective amount of a composition comprising a luminescent noble metal nanoparticle, wherein the surface of the noble metal nanoparticle is coated with a ligand, and wherein the noble metal nanoparticle is about 2 nm to 5 nm in diameter, wherein a portion of the noble metal is present as a radionuclide, wherein the ligand is capable of binding a tumor marker; and at least one therapeutic agent.

20. The method of claim 19, wherein the composition is administered orally, intravenously, nasally, subcutaneously, intramuscularly or transdermally.
